(19) United States
(12) Patent Application Publication
(10) Pub. No.: US 2018/0074032 A1
(43) Pub. Date: Mar. 15, 2018

(54) SYSTEM AND METHOD OF TESTING FOR AIRBORNE PATHOGENS, TOXINS, POLLUTANTS AND/OR CONTAMINANTS

(71) Applicant: Delta M Incorporated, Milton (CA)

(72) Inventor: Edward George Magee, Mississauga (CA)

(73) Assignee: Delta M Incorporated, Milton (CA)

(21) Appl. No.: 15/687,722

(22) Filed: Aug. 28, 2017

Related U.S. Application Data
(60) Provisional application No. 62/385,458, filed on Sep. 9, 2016.

(51) Int. Cl. G01N 33/00

(52) U.S. Cl. CPC G01N 33/0014; G01N 33/0062

(57) ABSTRACT

In a system and method of testing for airborne pathogens, toxins, pollutants and contaminants, a filter facility supplies a user with a filter member for use in an air handling unit. At the end of its service life, the filter member is removed from the unit, and sealed within a bag. The bag is packed in a shipping box supplied by, and returned to, the filter facility. Thereafter, one or more samples may be taken from the filter member, and the one or more samples may be tested for a wide array of pathogens, toxins, pollutants and/or contaminants. The test results are thereafter reported to the user. A refurbished/recycled filter member may optionally be supplied to the user for subsequent testing.

### FIELD OF THE INVENTION

The invention relates generally to the field of systems and methods of testing for airborne particles and, more particularly, to a system and/or method of testing for airborne pathogens, toxins, pollutants and/or contaminants.

### BACKGROUND OF THE INVENTION

In the prior art, air handling units have routinely been included as part of forced-air circulation heating, ventilation, and/or air-conditioning (“HVAC”) systems. A facility's air may circulate through the air filter(s) of a forced-air circulation system about once every day. As air filters become clogged, they tend to catch more particulate matter. In such a facility, it is be desirable to provide high indoor air quality.

In the prior art, mold inspections at such a facility can be very expensive, depending on the scale of the building being tested. It may be desirable to provide a more cost-effective solution. Moreover, in the prior art, it may not have even been feasible to perform air quality tests on multiple floors at the same time, given limitations on available equipment for testing purposes.

Some of the prior art may have included tape applied to the remove deposit from the wall of a facility, and duct work may have been swabbed post-filter.

All of the prior art may suffer from one or more shortcomings and/or disadvantages which may preferably be readily appreciable by and/or suggested to those skilled in the art in view of the teachings and/or disclosures hereof.

It may be desirable to provide a system and/or method of testing for airborne pathogens, toxins, pollutants and/or contaminants.

Preferably, one or more of the following advantages may be associated with and/or realized by use of the system and/or method according to the invention: (a) a cost-effective way of testing the user's air; (b) non-invasive testing methods; (c) simple, straight-forward kits for the user's technicians; (d) accredited third party non-biased laboratory results; (e) recommendations based on test report findings; (f) overall building health check from circulated air; and/or (g) improvement to the overall health of the user and the user's employees.

It is an object of the present invention to obviate or mitigate one or more disadvantages and/or shortcomings associated with the prior art, to meet or provide for one or more needs and/or advantages, and/or to achieve one or more objects of the invention—one or more of which may preferably be readily appreciable by and/or suggested to those skilled in the art in view of the teachings and/or disclosures hereof.

### SUMMARY OF THE INVENTION

According to the invention, there is disclosed a system and/or method of testing air filter members for airborne pathogens, toxins, pollutants and/or contaminants. In the system and method, a filter facility supplies a user with a test air filter member for use in an air handling unit. The test air filter member preferably may bear one or more markings or other indicia to indicate it for later testing or to provide supplemental information to facilitate such tracking.

After a specified period of air filter usage, which may be the normal service life of the test air filter member (or some shorter period), the test air filter member may be removed from the air handling unit, and preferably sealed, in airtight relation, within a sealable plastic bag. The test air filter member, preferably sealed within the plastic bag, may be packed in a shipping box (or shipping envelope) supplied by the filter facility, and returned to the filter facility. When the test air filter member arrives back at the filter facility, the test air filter member preferably may be taken to a partitioned clean portion of the filter facility which provides a sterile environment for sampling the test air filter member, if necessary or appropriate depending in part on a nature of the test air filter member to be tested. After sampling, if necessary, the sample from the test air filter member—and/or the test air filter member itself—is delivered to a laboratory facility for testing. At the laboratory facility, the sample and/or test air filter member is submitted to standardized testing for a wide array of pathogens, toxins, pollutants and/or contaminants. After testing, the laboratory facility returns the test results to the filter facility, in a standardized format. The filter facility conducts a further analysis of the test results for reporting to the user.

According to an aspect of the invention, the air handling unit preferably may be part of a forced-air circulation HVAC system.

According to one aspect of the invention, the test air filter member may be a test air filter insert and/or patch which is supplied to the user, preferably along with and/or removably attached to a test air filter. According to one aspect of the invention, the test air filter insert and/or patch may be applied and/or attached to a test portion of the test air filter. The test portion preferably may be predetermined and located in the same spot on the test air filter every time. Preferably, the test portion may comprise a center area of the test air filter. According to one aspect of the invention, an insert and/or patch guide preferably may be provided, which preferably may be sized and adapted to accommodate the test air filter insert and/or patch on the test air filter. The insert and/or patch guide preferably may clearly and consistently indicate the same test portion of any test air filter from which the test air filter insert and/or patch preferably may be taken every time. According to one aspect of the invention, the test air filter insert and/or patch may preferably be removed from (the test air filter and) the air handling unit at the end of its service life. The service life of the test air filter insert and/or patch preferably may be shorter than a comparable service life of the test air filter. (In some embodiments according to the invention, the service life of the test air filter insert and/or patch may be about one day or less, e.g., possibly as short as about 5 to 10 minutes.) The plastic bag may be provided in the form of a plastic bag envelope. After removal from the air handling unit, the test air filter insert and/or patch may be sent back to the filter facility in the plastic bag envelope alone, or in the plastic bag inside the shipping box or shipping envelope. When the test air filter insert and/or patch arrives back at the filter facility, it may be delivered directly to the laboratory facility for testing.

According to another aspect of the invention, however, the test air filter member may be an entire test air filter, (i.e., including frame, central filter media, peripheral sealing means etc.), provided without any said test air filter insert and/or patch.

According to yet another aspect of the invention, the test air filter preferably may be a reusable and recyclable air filter, and/or the filter facility preferably may be a filter processing and refurbishment facility, whereat other air filters preferably may be refurbished and/or recycled.

According to one aspect of the invention, the test air filter member preferably may be supplied to the user, sealed within a plastic bag (e.g., a cellophane bag), with instructions for in situ installation. According to one aspect of the invention, the sealable plastic bag preferably may be re-sealable, and the same plastic bag in which the test air filter member had been sealed when it was originally supplied to the user.

According to one aspect of the invention, the markings or other indicia may preferably make the test air filter member (and/or any accompanying test air filter) easily distinguishable from other filters (and parts thereof), and/or they may comprise a red frame and/or another coloring, statement and/or indicia to indicate it for later testing and may include supplemental information to facilitate the tracking of the test air filter member.

According to one aspect of the invention, the test air filter member preferably may be installed in, and removed from, the center of a bank of filters in the air handling unit. The bank of filters preferably may be removed from the air handling unit and, along with the test air filter member, returned to the filter facility once about every three (3) months or so, or on demand.

According to one aspect of the invention, the filter facility preferably supplies the test air filter member within the shipping box, when it originally supplies the test air filter member to the user.

According to another aspect of the invention, when the bank of filters is removed from the air handling unit, the test air filter member preferably (sealed within the plastic bag) may be packed into the shipping box along with the others in the removed bank of filters and they preferably may be together returned to the filter facility in the same shipping box.

According to one aspect of the invention, the service life of the test air filter preferably may be about three (3) months or so.

According to an aspect of the invention, another set of air filters is supplied by the filter facility for swapping into the air handling unit when the bank of filters is removed therefrom.

According to one aspect of the invention, a single sample preferably may be taken from the collection mat of the test air filter. The sample preferably may comprise dirt, debris and/or other material which collected on the test air filter over its service life.

According to one aspect of the invention, in the clean portion of the filter facility, at least a test portion of the test air filter preferably may be swabbed. Preferably, a pre-sterilized disposable swab, supplied with its own pre-sterilized test tube storage cylinder, may be used.

According to one aspect of the invention, the swabbed sample preferably may be taken from a collection mat of the test air filter, where the test portion preferably may be located.

According to one aspect of the invention, the test portion of the test air filters preferably may be predetermined and the test air filters preferably may be swabbed from the same spot every time. Preferably, the test portion may comprise a center area of the test air filter.

According to one aspect of the invention, a frame guide, which preferably may be sized and adapted to fit over a frame of the test air filter, preferably may be used by the filter facility to swab the same spot every time as aforesaid. The frame guide preferably may be shaped to define a cut-out therein, which preferably may clearly and consistently indicate the same test portion of the test air filter which preferably may be swabbed every time. The frame guide may preferably comprise a plastic template of a standard size (e.g., 15 cm×15 cm) which preferably may be placed on the middle surface of the test air filter.

According to one aspect of the invention, the swab preferably may be brushed against the test air filter's surface, preferably keeping within a perimeter of the frame guide.

According to one aspect of the invention, preferably after swabbing the test portion of the test air filter, the exposed swab preferably may be sealed within the pre-sterilized test tube storage cylinder for delivery to the laboratory facility. The storage cylinder preferably may be labelled with date, time, filter code and/or sampler initials.

According to one aspect of the invention, after sampling, the test air filter preferably may be kept by the filter facility, preferably in a preserved state, for another three (3) months or more.

According to one aspect of the invention, the array of pathogens, toxins, pollutants and/or contaminants—for which the sample preferably may be tested at the laboratory facility—preferably may include: (A) molds, bacteria and/or yeast; (B) one or more that typically may be found in commercial, industrial, governmental, school, residential and/or environmental contexts; and/or (C) one or more that typically may become entrained in the filter mats by way of regular and/or forced circulation of air through the air handling unit in a building.

According to one aspect of the invention, the filter facility may preferably establish and/or record a test filter log on a secure IT Server, and may preferably track where each test air filter was placed and for how long. The use of machine readable optical codes, as discussed below, is particularly useful to efficiently facilitate such tracking.

According to one aspect of the invention, the filter facility may preferably conduct on-going analyses and/or monitoring of test air filter members returned from the user.

According to one aspect of the invention, the filter facility may preferably establish a baseline over time, and/or graph information/data from its analysis quarterly and/or seasonally. A seasonal baseline preferably may be established after about twelve (12) to sixteen (16) months, and/or a seasonal comparison of the user's same building(s) preferably may be performed over time.

According to one aspect of the invention, the filter facility may preferably provide a graphical report on its analysis of the test results to the user. The report may also include comparison test results from earlier test of the same air handling unit or from said previously established baseline.

According to one aspect of the invention, the filter facility may preferably charge the user for this testing service on a subscription model. Preferably, the subscription model may be on a three-year contract.

Other advantages, features and characteristics of the present invention, as well as methods of operation and functions of the related elements of the structure, and the combination of parts and economies of manufacture, will become more apparent upon consideration of the following detailed description.

Additionally, novel features which are believed to be characteristic of the system and method according to the present invention, as to its structure, organization, use and method of operation, together with further objectives and advantages thereof, may be better understood from figures in which one or more preferred embodiments of the invention may later be illustrated by way of example. It is expressly understood, however, that any such figures will be for the purpose of illustration and description only, and not intended as a definition of the limits of the invention.

### DETAILED DESCRIPTION OF VARIOUS EMBODIMENTS

In a system and/or method of testing for airborne pathogens, toxins, pollutants and/or contaminants, according to one or more preferred embodiments of the invention, a filter facility supplies a user with a test air filter member for use in an air handling unit. The filter facility preferably may be a filter processing and refurbishment facility, whereat other air filter members preferably may be refurbished and/or recycled. The test air filter member preferably may be a reusable and recyclable test air filter. The test air filter may be supplied to the user, sealed within a plastic bag (e.g., a cellophane bag), with instructions for in situ installation. The test air filter preferably may be installed in, and removed from, the center of a bank of filters in the air handling unit. The air handling unit preferably may be part of a forced-air circulation HVAC system.

According to the invention, the test air filter preferably may bear one or more markings or other indicia to indicate it for later testing. The markings or other indicia make the test air filter member easily distinguishable from other filters. The markings or other indicia may include a red frame and/or another coloring, a prominent notice indicating it as a test air filter member, and/or other indicia to indicate it for later testing. Such indicia may also advantageously include supplemental information, such as, by way on non-limiting example, the identity of the user to whom the test air filter is supplied, the location and/or type of the air handling unit for which the test air filter is being supplied, the date on which the test air filter was supplied, and/or an indication of the service life of the test air filter. Such supplemental information may be partially, or fully, in the form of machine readable optical codes, being for example, UPC bar codes, or Quick Response (often abbreviated as “QR”) codes to facilitate the use of electronic scanning equipment during shipping and handling by the filter facility, laboratory facilities, or institutional end users during shipping and handling of the test air filters as described more fully hereinbelow.

According to the invention, when the test air filter member reaches the end of its service life, the test air filter member is removed from the air handling unit, and sealed, in airtight relation, within a sealable plastic bag. The service life of the test air filter preferably may be about three (3) months or so. The bank of filters may be removed from the air handling unit and, along with the test air filter, returned to the filter facility once about every three (3) months or so, or on demand. Another set of air filters preferably may be supplied by the filter facility for swapping into the air handling unit when the bank of filters is removed therefrom.

The sealable plastic bag preferably may be re-sealable, and the same plastic bag in which the test air filter had been sealed when it was originally supplied to the user.

In some (alternate) embodiments of the invention, the sealable plastic bag preferably may be provided with a removable internal bag liner formed of a plastic material and/or a filter material. In such embodiments, loose particulate matter may become dislodged from the filter and end up on the bag liner. In some such embodiments of the invention, the bag liner preferably also may be removed from the plastic bag and one or more samples therefrom may be submitted for testing along with the filter.

The test air filter, preferably sealed within the plastic bag, may be packed in a shipping box supplied by the filter facility, and returned to the filter facility. The filter facility preferably supplied the test air filter within the shipping box, when it originally supplied the test air filter to the user, and return labels pre-addressed to the filter facility are also preferably contained within the shipping box.

In some (alternate) embodiments of the invention, instead of providing a separate sealable plastic bag, the shipping box may be provided with a plastic liner and, when the test air filter reaches the end of its service life, the test air filter may be removed from the air handling unit and sealed, in airtight relation, within the shipping box (without any separate plastic bag).

Preferably, however, when the bank of filters is removed from the air handling unit, the test air filter (sealed within the plastic bag) is packed into the shipping box along with the others in the removed bank of filters and they are together returned to the filter facility in the same shipping box using the aforesaid labels pre-addressed to the filter facility.

When the test air filter arrives back at the filter facility, the test air filter preferably may be taken to a partitioned clean portion of the filter facility which provides a sterile environment for sampling the test air filter. Masks and gloves are preferably worn by personnel in the clean portion of the filter facility. In the clean portion of the filter facility, at least a test portion of the test air filter is swabbed. Preferably, a pre-sterilized disposable swab, supplied with its own pre-sterilized test tube storage cylinder, may be used. The swabbed sample is taken from a collection mat of the test air filter, where the test portion is located.

Preferably, the test portion of the test air filters is predetermined and the test air filters are swabbed from the same spot every time. Preferably, the test portion comprises a center area of the test air filter. A frame guide, which is sized and adapted to fit over a frame of the test air filter, is used by the filter facility to swab the same spot every time as aforesaid. The frame guide is shaped to define a cut-out therein, which clearly and consistently indicates the same test portion of the test air filter which is to be swabbed every time. The frame guide may preferably comprise a plastic template of a standard size (e.g., 15 cm×15 cm) which is placed on the middle surface of the test air filter. Keeping within the perimeter of the frame guide, the swab preferably may be brushed against the test air filter's surface for 5 measured seconds.

Preferably, a single sample is taken from the collection mat of the test air filter (although multiple samples may be taken in alternate embodiments of the invention). The sample preferably comprises dirt, debris and/or other material which collected on the test air filter over its service life. After swabbing the test portion of the test air filter, the exposed swab is sealed within the pre-sterilized test tube storage cylinder for delivery to the laboratory facility.

Preferably, but not necessarily, the swab is placed in the pre-sterilized test tube storage cylinder without any (further) stabilization media. Instead of the pre-sterilized test tube storage cylinder, other specialized packaging may be used for delivery of the sample to the laboratory facility. The storage cylinder is preferably labelled with date, time, filter code and/or sampler initials.

In some embodiments of the invention, a swab sample may also (or instead) be taken, as a reference, from a clean test air filter.

After sampling, the test air filter preferably may be kept by the filter facility, preferably in a preserved state, for another three (3) months or more.

After sampling, the sample from the test air filter is delivered to a laboratory facility for testing. Samples preferably may be delivered individually (or potentially en masse) to the laboratory facility. Alternately, the sample(s) may be picked-up by the laboratory facility on a regular basis.

At the laboratory facility, the sample is submitted to standardized testing for a wide array of pathogens, toxins, pollutants and/or contaminants. The array of pathogens, toxins, pollutants and/or contaminants preferably may include molds, bacteria and/or yeast. The laboratory facility may count a quantity of mold colonies found on Petri dishes. The laboratory facility may determine a prevalence of mold.

Please see the section entitled “Additional Notes”, appended below, for further information concerning some of the tests which may be performed, and various other aspects of the system and/or method according to the present invention.

The array of pathogens, toxins, pollutants and/or contaminants preferably may include one or more that typically may be found in commercial, industrial, governmental, school, residential and/or environmental contexts. The array of pathogens, toxins, pollutants and/or contaminants preferably may include one or more that typically may become entrained in the filter mats by way of regular and/or forced circulation of air through the air handling unit in a building.

After testing, the laboratory facility returns the test results to the filter facility, in a standardized format.

The filter facility conducts a further analysis of the test results for reporting to the user. The filter facility provides its reports to the user's designated contact. The filter facility establishes and records a Test Filter Log on a secure IT Server, and tracks where each test air filter was placed and for how long.

The filter facility conducts on-going analysis and monitoring of test air filters returned from the user. The filter facility establishes a baseline over time, and graphs information/data from its analysis quarterly and/or seasonally. A seasonal baseline preferably may be established after about 12-16 months. A seasonal comparison of the user's same building(s) is performed over time.

The filter facility provides a graphical report on its analysis of the test results to the user. Information/data may be graphed quarterly. The graphical report preferably may comprise performance data concerning the test air filter(s) received from the user and may include comparison test results from earlier tests run for the same air handling unit.

The filter facility preferably charges the user for this testing service on a subscription model. Preferably the subscription model is on a three-year contract. Alternately, the charge may be made as a separate/individualized fee to the user, or incorporated into the cost of a single-use filter.

To put some of the foregoing another way, one filter may be tested. The filters may be taken out, and the one marked for testing may be put into a special bag. That bag may be shipped back to the filter facility in the same box as the other filters. When the filters arrive back at the facility, the test filter may be taken to a partitioned clean portion of the facility which provides a sterile environment for testing. There, the test filter may be swabbed. A pre-sterilized disposable swab may be used, preferably one supplied with its own test tube storage cylinder. After swabbing the test filter, the swab may be placed in its test tube storage cylinder for delivery to the laboratory facility.

Preferably, the test filters are swabbed from the same spot every time, preferably, from the center portion of the test filters. The facility may have a customized frame guide on hand to facility same, for example, a frame guide that fits over the frame, with a cutout therein, to clearly indicate to the facility personnel that portion of the test filter which should be swabbed.

One of the potential advantages of the invention may be to reduce or curtail the spread of sickness, and/or to destroy bacteria.

Preferably, according to the invention, mold counts may be taken on HVAC air filters, preferably to improve management of air quality for users, and/or to provide on-going monitoring of mold within buildings in a cost-effective manner.

Various different test scenarios may be contemplated according to the present invention, for example, inside air versus outside air, one building versus another in the same area, and/or a seasonal comparison of the same building over time.

According to one preferred embodiment of the invention, a number of steps may be performed as follow:

1. Mark and codify, preferably with machine readable optical codes, a group of HVAC filters (aka Test Filters) for air sampling purposes. Preferably, the marking may make these filters easily distinguishable from normal filters. A Test Filter Log preferably may be established and secured on an IT Server. A unique code preferably may enable the filter facility to track where the Test Filter was placed and for how long.

2. A sampler preferably wears a mask and gloves. A plastic template of a standard size (e.g., 15 cm×15 cm) preferably may be provided and placed on a middle surface of the Test Filter. A swab sample of a clean Test Filter preferably may be taken. Keeping within the perimeter of the template, the swab preferably may be brushed against the filter's surface for 5 measured seconds. The exposed swab preferably may be sealed in a sampling tube. The sampling tube preferably may be labeled with date, time, filter code and sampler initials. The Test Filter Log preferably may be updated to cross-reference sample number with Test Filter number. Preferably, according to some preferred embodiments of the invention, for a baseline, one sample in ten may be sent to the lab for testing.

3. The Test Filter preferably may be sealed in a cellophane bag with instructions for in situ installation. The Test Filter Log preferably may be updated.

4. Preferably, after a Test Filter may be added to a user order, the filter code in the Test Filter Log may be updated with date, time, user address, and user order—n.b., in some preferred embodiments of the invention, it may be required and/or desirable that the Test Filter not be used to filter outside air. In some such embodiments, it may be desirable and/or required to limit the Test Filter to recycled air in a closed air system.

5. After a specified duration (e.g., about 3 or 4 months according to some preferred embodiments of the invention) the user's used filters may be replaced. The soiled Test Filter may be bagged, sealed and added with the lot being returned to the filter facility.

6. When the soiled Test Filter is received at the filter facility, two samples preferably may be taken using the same methodology used in step 3, above. One sample may be stored as a reference sample. The second sample may be sent to the lab for testing.

7. According to some preferred embodiments of the invention, the lab test preferably may count the quantity of mold colonies found on the Petri dishes—n.b., classification of molds and their source(s) may be outside the scope of the tests according to some preferred embodiments of the invention.

8. After about 12-16 months, a seasonal baseline from these test results may be established.

9. A graphical report of the test results may be presented to the user.

Much of the preceding description may concern a test air filter according to one preferred embodiment of the invention. The present invention, however, is not so limited. In other preferred embodiments of the invention, a test air filter insert and/or patch may be supplied to the user. It may be supplied to the user along with and/or removably attached to a test air filter.

Preferably, in this particular embodiment of the invention, the test air filter insert and/or patch may be applied and/or attached to a test portion of the test air filter. The test portion preferably may be predetermined and located in the same spot on every test air filter. Preferably, the test portion may be a center area of the test air filter.

An insert and/or patch guide preferably may be provided in this preferred embodiment of the invention. It preferably may be sized and adapted to accommodate the test air filter insert and/or patch on the test air filter. The insert and/or patch guide preferably may clearly and consistently indicate the same test portion of every test air filter from which the test air filter insert and/or patch preferably may be taken every time.

In this particular embodiment of the invention, the test air filter insert and/or patch may preferably be removed from (the test air filter and) the air handling unit at the end of its service life. The service life of the test air filter insert and/or patch preferably may be shorter than a comparable service life of the test air filter. (The service life of the test air filter insert and/or patch may be about one day or less, e.g., possibly as short as about 5 to 10 minutes.)

The plastic bag may be provided in the form of a plastic bag envelope in this preferred embodiment of the invention. After removal from the air handling unit, the test air filter insert and/or patch may be sent back to the filter facility in the plastic bag envelope alone. Or, the shipping box may be provided in the form of a shipping envelope, the test air filter insert and/or patch may be sent back to the filter facility in the plastic bag inside the shipping envelope. When the test air filter insert and/or patch arrives back at the filter facility, it may be delivered directly to the laboratory facility for testing.

Preferably, one or more various embodiments of the system and/or method according to the present invention may provide a simple and cost effective way to determine if harmful molds, bacteria and/or yeast may be present in the air a user breathes, and how prevalent the levels of mold are. The testing methods according to the present invention may be minimally invasive in the user's buildings.

The filter facility's filters, which may be selected to be included in the testing, may include a sampling kit with return labels. This will allow the user's technician to isolate an interior HVAC air filter, from the center of the air handling unit, and safely seal it inside the included bag with tags, to prevent any contamination, while also identifying the filter to be tested after return to the filter facility.

Preferably, the mold, yeast and bacteria testing which may be performed according to the invention may provide lab quality results in testing the user's air, preferably, using the same filter facility that already otherwise ensures the quality and cleanliness of the user's recycled filters, preferably to test swab samples from the selected returned air filters with lab quality results.

### Additional Notes

Microbiology laboratories may be used for some of the tests which may be performed according to the invention.

It may be desirable to test for bacteria, and some of the variables in such tests, according to the invention, may include (i) the sampling process and handling of samples (e.g., swabbing the surface of filtration media and other techniques); (ii) storage and longevity of reference samples; (iii) the type(s) of airborne bacteria for which the tests may be performed; and (iv) the potentially concurrent/multiplex nature of a single test for multiple forms of bacterial.

It may be desirable to test for molds, e.g., by spore count, e.g., by visual count using microscopy.

It may be desirable for these and other tests to be performed on air filters which have been in situ for about 4-6 months.

Human health may be influenced by the presence of dead and/or living mold spores. A spore count test may be performed without distinguishing between dead and alive spores, e.g., by providing a total count of both dead and alive spores, preferably to see the cumulative impact over the period in question.

For total spore counts, it may be desirable to collect air samples, e.g., without swabbing the test air filters. To perform a spore count test, a test air filter insert may be shipped without, e.g., a cooler box or ice pack.

Among other things, the death rate of spores may be influenced by the presence of UV light, the level of humidity (e.g., dry air may accelerate the death rate), and the types of mold spore to be tested.

Other testing may be performed for live molds, e.g., using a petri dish. Petri dish tests may be focused on counting live molds and identifying one or more species of mold.

Also or instead, it may be desirable to ascertain what may have settled on the filters and, in doing so, the filters may be swabbed and the swabs may be analyzed, e.g., by culturing on agar and/or by direct microscopy.

After several months of filtering air, there may be a significant build-up of dirt, dust and/or mold on the filter media. A test air filter insert and/or patch (e.g., 4 cm×4 cm) may be provided, which a laboratory might end up using to perform a spore count test (even though there may be a significant level of dirt on the surface area being tested).

The insert or patch may or may not be swabbed, before culturing and testing for colony forming units per unit area, e.g., to better detect spores which otherwise might be mixed with dust and/or difficult to see via direct microscopy.

According to some preferred embodiments of the invention, it may be preferable to detect dead spores via visual microscopy, rather than with culturing, e.g., to better estimate contamination.

For some of the tests which might be performed, e.g., mold spore count, it may be desirable for the filter test substrate (i) to have low, reduced and/or minimal levels of dirt and/or (ii) to only be exposed for about a day or so. Design, administration and/or control of a mold testing process, according to the invention, may be adjusted accordingly as may be obvious to persons skilled in the art in view of the teachings and disclosures herein.

In a direct microscopic examination, samples may be checked visually and scanned under a microscope for mold growth. A qualitative assessment of mold growth may be performed if/where possible. Areas showing mold growth may be sampled and observed under a microscope at different magnifications if/as appropriate.

Bacteria testing may be similar to mold testing in some respects. Bacteria, like mold, may always be in the air. A total bacteria count can be done. Some of the variables in bacteria testing may include: (a) interpretation of results, and (b) threshold numbers tested.

Results may be interpreted comparatively, e.g., by comparing multiple rooms, floors and/or buildings, and/or by performing a comparison over time, e.g., from month to month and/or relative to some event, e.g., a mediation event.

General or total bacteria count tests may be performed, and specific bacteria of interest may be identified, e.g., by performing agar-based tests for specific target organisms, or such other tests as may be available.

In a total bacteria count test, samples may be swabbed, and the swabs vortexed to release bacteria from the swab tip. Later, a number of colonies may be counted and/or estimated after incubation.

In some of the tests which may be performed according to the invention, it may be desirable to have specific protocols for sampling and handling of samples, e.g., to keep the sample media from becoming overly blanketed by mold or bacteria by limiting exposure to, e.g., air for a quantified or quantifiable period of time, and/or a quantified or quantifiable volume of air. For example, for some of the tests to be performed, it may be desirable to limit air exposure to about 5 to 10 minutes, or to take an air sample of perhaps about 140 to 150 liters of air over about 5 to 6 minutes.

It may be desirable to determine or quantify a volume of air flowing past the filter over a specific period of time period, e.g., to determine the number of bacteria colonies per cubic meter of air.

According to some preferred embodiments of the invention and perhaps depending on the types of tests to be performed, it may be desirable, in handling samples, to return each sample to a laboratory within about 48 hours, or perhaps within about 2 to 3 days.

If desired and/or required, samples may be stored in cooler boxes with ice packs, or on ice, to extend longevity.

According to some preferred embodiments of the invention and perhaps depending on the types of tests to be performed, it may be preferable for samples to be delivered to the laboratory shortly after receipt by the processing facility.

Some of the tests which may be performed according to the invention may include one or more of the following: mold tests, yeast tests, algae tests, coal ash tests, asbestos tests, allergen tests (e.g., animal dander), bacteriological tests, and industrial hygiene tests for certain materials.

The foregoing description has been presented for the purpose of illustration and is not intended to be exhaustive or to limit the invention to the precise form disclosed.

Naturally, in view of the teachings and disclosures herein, persons having ordinary skill in the art may appreciate that alternate designs and/or embodiments of the invention may be possible (e.g., with substitution of one or more components, features, steps for others, with alternate configurations of components, features, steps). Although some of the components, relations, configurations, features and/or steps according to the invention are not specifically referenced in association with one another, they may be used, and/or adapted for use, in association therewith. All of the aforementioned, depicted and various structures, configurations, features, relationships, steps, utilities and the like may be, but are not necessarily, incorporated into and/or achieved by the invention. Any one or more of the aforementioned structures, configurations, features, relationships, steps, utilities and the like may be implemented in and/or by the invention, on their own, and/or without reference, regard or likewise implementation of any of the other aforementioned structures, configurations, features, relationships, steps, utilities and the like, in various permutations and combinations, as will be readily apparent to those skilled in the art, without departing from the pith, marrow, and spirit of the disclosed invention.

Other modifications and alterations may be used in the design, manufacture, and/or implementation of other embodiments according to the present invention without departing from the spirit and scope of the invention, which is limited only by the claims of any regular patent applications claiming priority herefrom.

I claim:

1. A method for testing for airborne pathogens, toxins, pollutants and/or other contaminants comprising:
a) supplying a user with a test air filter member for use in an air handling unit;
b) removing the used test air filter member from the air handling unit after a period of usage therein;
c) sealing the used test air filter member in airtight relation within a sealable container;
d) placing the used test air filter member within the sealable container into a shipping container;
e) returning the used test air filter member in the shipping container to a filter facility;
f) removing the test air filter member from the shipping container and from within the sealable container at the filter facility;
g) taking samples of airborne pathogens, toxins, pollutants and/or other contaminants from the test air filter member;
h) testing the samples from step g) for pathogens, toxins, pollutants and/or other contaminants; and
i) reporting the test results to the user.

2. The method of claim 1, wherein one or both of steps g) and h) are carried out within a partitioned clean portion of the filter facility which provides a sterile environment.

3. The method of claim 1, wherein the samples taken in step g) are delivered to a laboratory facility for conducting step h) thereat, and additionally comprising, before step i), sub-step h)¹ having the laboratory facility return the test results to the filter facility for reporting by the filter facility.

4. The method of claim 3, additionally comprising, after sub-step h)¹, sub-step h)², wherein the filter facility conducts a further analysis of the test results received from the laboratory facility before reporting the test results to the user in step i).

5. The method of claim 4, wherein the filter facility reports the test results to the user in the form of a graphical report.

6. The method of any one of claim 4, wherein the filter facility reports the test results to the user accompanied with comparison test results.

7. The method of any one of claim 1, wherein the air handling unit is part of a forced-air circulation HVAC system.

8. The method of any one of claim 1, wherein the user is supplied in step b) with a test air filter member bearing one or more markings/indicia to identify it for later testing.

9. The method of claim 8, wherein said markings/indicia additionally comprises information to uniquely identify said user.

10. The method of claim 9, wherein said markings/indicia additionally comprises information to uniquely identify the type and location of said air handling unit.

11. The method of any one of claim 8, wherein said markings/indicia comprises one or more machine readable optical codes.

12. The method of any one of claim 1, wherein the air handling unit is a forced-air circulation HVAC system and the test air filter member is a test air filter for use in said HVAC system.

13. The method of claim 12, wherein the test air filter member comprises a test air filter insert/patch removably attachable to the test air filter.

14. The method of claim 13, wherein the test air filter insert/patch is removably attachable to a test portion of the test air filter positioned in a predetermined location on said test air filter.

15. The method of claim 14, wherein said predetermined location of the test portion comprises a central area of the test air filter.

16. The method of claim 15, wherein said central area is demarcated on the test air filter.

17. The method of any one of claim 14, wherein step b) is modified to carry out removal of the test air filter insert/patch from the test portion of the test air filter and all steps thereafter are carried out in relation to the insert/patch removed in step b).

18. The method of any one of claim 1, wherein step b) and all steps thereafter are carried out after the end of the service life of the test air filter insert and/or patch.

19. The method of any one of claim 1, wherein the test air filter member is supplied in step a) within a shipping container suitable for re-use as the shipping container in step d).

20. The method of any one of claim 1, wherein the test air filter member supplied in step a) is supplied within a sealable container adapted for re-use as the sealable container in step c).

21. The method of claim 20, wherein the sealable container is a re-sealable plastic bag.

22. The method of any one of claim 1, wherein return labels pre-addressed to the filter facility are additionally supplied to the user in step a).

23. The method of any one of claim 22, wherein instructions for in situ installation and use of the air filter member and for return of the air filter member to the facility are additionally supplied to the user in step a).

24. The method of any one of claim 1, wherein the filter facility is a filter processing and refurbishment facility, and wherein the method additionally comprises, after step i), repeating the cycle of steps a) through i) one or more times, with step a) being modified in subsequent cycles to supplying the user with a refurbished and/or recycled test air filter member.

25. The method according to claim 24, wherein the cycle of steps a) through i) is repeated approximately once every three (3) months.
